(19) United States
(12) Patent Application Publication
(10) Pub. No.: US 2020/0078459 A1
(43) Pub. Date: Mar. 12, 2020

(54) NEAR INFRARED (NIR) PHOTODYNAMIC THERAPY (PDT) IN COMBINATION WITH CHEMOTHERAPY

(71) Applicant: Health Research, Inc., Buffalo, NY (US)

(72) Inventors: Ravindra K. PANDEY, East Amherst, NY (US); Farukh DURRANI, East Amherst, NY (US); Khurshid GURU, East Amherst, NY (US)

(21) Appl. No.: 16/469,581

(22) PCT Filed: Dec. 13, 2017
(86) PCT No.: PCT/US17/66122

§ 371 Date: Jun. 13, 2019

Related U.S. Application Data
(60) Provisional application No. 62/433,550, filed on Dec. 13, 2016.

(51) Int. Cl. A61K 41/00; A61N 5/06; A61K 49/00

(52) U.S. Cl. CPC A61K 41/0057; A61N 2005/0659; A61K 49/0036; A61N 5/062

(57) ABSTRACT

Methods for treatment of cancer are provided. The methods are based on the use of both NIR photosensitizer(s) and chemotherapy agent(s). The NIR photosensitizers have a tetrapyrrolic core or reduced tetrapyrrolic core. Also provided are pharmaceutical compositions comprising NIR photosensitizer(s) and chemotherapy agent(s). Also provided are kits comprising NIR photosensitizer(s) and chemotherapy agent(s) and instructions for their use.

### CROSS REFERENCE TO RELATED APPLICATIONS

This application claims priority to U.S. Provisional Application No. 62/433,550, filed on Dec. 13, 2016, the disclosure of which is hereby incorporated herein by reference.

### FIELD OF THE DISCLOSURE

The disclosure generally relates to methods of using a combination of photosensitizers and chemotherapy agents to treat cancer. More particularly, the disclosure relates to methods of using a combination of near infrared (NIR) photosensitizers and chemotherapy agents to treat cancer.

### BACKGROUND OF THE DISCLOSURE

The rationale for combination therapy is to use two or more drugs that work by different mechanisms in combination. Photodynamic therapy (PDT), a loco-regional treatment is a three component treatment modality in which the tumor-avid photosensitizer (non-toxic by itself), on exposing with light react with molecular oxygen present in tumor and generates highly cytotoxic reactive oxygen species (singlet oxygen, ¹O₂), which destroys the tumor vasculature leading to tumor destruction. Photodynamic therapy has shown great promise in treating a variety of tumors, which can be accessed with light. Unfortunately, it is not an effective treatment modality for patients suffering with tumor metastases.

### SUMMARY OF THE DISCLOSURE

The present disclosure provides methods of treatment based on the use of a combination of NIR photosensitizer(s) and chemotherapy agent(s). The present disclosure also provides kits comprising NIR photosensitizer(s) and chemotherapy agent(s), and instructions for use of the NIR photosensitizer(s) and chemotherapy agent(s) (e.g., use in methods of the present disclosure.

In an aspect, the present disclosure provides methods of treatment. The methods are based on the use of a combination of NIR photosensitizer(s) and chemotherapy agent(s). The methods can be used to treat cancer in an individual. The methods can be referred to as combination treatments.

In an example, a method for treating an individual in need of treatment for cancer comprises: administering to the individual one or more NIR photosensitizer comprising a tetrapyrrolic core or reduced tetrapyrrolic core (e.g., an effective amount of one or more such NIR photosensitizer); administering of one or more chemotherapy agent (e.g., an effective amount or a sub-therapeutic amount of one or more chemotherapy agent); and irradiating the individual with electromagnetic radiation having a wavelength of 650 nm to 800 nm. In an example, the amount of chemotherapy agent is effective to reduce tumor size without significant toxicity. In an example, an effective amount of one or more NIR photosensitizer and an effective amount of one or more chemotherapy agent are administered to the individual. In another example, an effective amount of one or more NIR photosensitizer and a sub-therapeutic amount of one or more chemotherapy agent are administered to the individual.

In an aspect, the present disclosure provides pharmaceutical compositions comprising one or more NIR photosensitizer and one or more chemotherapy agent. The compositions may comprise one or more pharmaceutically acceptable carrier.

In another aspect, the present disclosure provides kits. In an example, a kit comprises NIR photosensitizer(s) and chemotherapy agent(s) and instructions for their use. In another example, a kit further comprises Bacillus Calmette-Guerin (BCG) vaccine.

### BRIEF DESCRIPTION OF THE FIGURES

For a fuller understanding of the nature and objects of the disclosure, reference should be made to the following detailed description taken in conjunction with the accompanying figures.

FIG. 1 shows relative absorption (solid line) and fluorescence (dotted line) spectra of HPPH (Photochlor) and Photobac in methanol at 5 μM. Both compounds in the presence of BSA (bovine serum albumin) or HSA (human serum albumin) produced a red shift of 5 nm with a broad NIR absorption at 665 and 787 nm. Therefore for in vivo PDT, the light treatment with HPPH and Photobac was performed at 665 and 787 nm respectively.

FIG. 2 shows a PDT response and antitumor activity of combination therapy of HPPH−PDT, Cisplatin alone and HPPH−PDT+Cisplatin weekly×3 dose (1 hour post PDT) in SCID mice bearing NSCLC lung cancer xenografts.

FIG. 3 shows a PDT response and antitumor activity of combination therapy of HPPH−PDT, Doxorubicin alone and HPPH+PDT+Doxorubicin weekly×3 dose (1 hour post PDT) in SCID mice bearing NSCLC lung cancer xenografts.

FIG. 4 shows a PDT response and antitumor activity of combination therapy of Photobac-PDT+Doxorubicin weekly×3 dose (1 hour post PDT) in SCID mice bearing NSCLC lung cancer xenografts.

FIG. 5 shows a PDT response and antitumor activity of combination therapy of HPPH−PDT, Irinotecan alone and HPPH−PDT+Irinotecan weekly×4 dose (1 hour post PDT) in SCID mice bearing FaDu head and neck cancer xenografts.

FIG. 6 shows a PDT response and antitumor activity of combination therapy of Photobac-PDT+Doxorubicin weekly×3 dose (1 hour post PDT) in SCID mice bearing FaDu head and neck cancer xenografts.

FIG. 7 shows a comparative long-term tumor response of SCID mice bearing UMUC3 tumors: BCG alone, HPPH−PDT and the combination of HPPH−PDT with BCG (for details see the text).

FIG. 8 shows PDT response and antitumor activity of combination therapy of HPPH−PDT, BCG alone and HPPH−PDT+BCG weekly×3 dose (1 hour post PDT) in SCID mice bearing T24 bladder cancer xenografts.

FIG. 9 shows individual Tumor Response: PDT response and Antitumor activity of combination therapy of HPPH 0.47 umol/kg+PDT+BCG (2×10e6) weekly×3 doses (1 hour post PDT) in SCID mice bearing UMUC-3 Urinary Bladder cancer tumors.

FIG. 10 shows individual Tumor Response: PDT response and Antitumor activity of combination therapy of HPPH 0.47 umol/kg+PDT+BCG (2×10e6) weekly×3 doses (1 hour post PDT) in SCID mice bearing T 24 Urinary Bladder cancer tumors.

FIG. 11 shows individual Tumor Response: PDT response and antitumor activity of combination therapy of HPPH 0.47 umol/kg+PDT in SCID mice bearing UMUC-3 Urinary Bladder cancer tumors.

FIG. 12 shows individual Tumor Response: PDT response and antitumor activity of combination therapy of HPPH 0.47 umol/kg+PDT in SCID mice bearing T24 Urinary Bladder cancer tumors.

FIG. 13 shows PDT response and antitumor activity of combination therapy of HPPH+PDT+Cisplatin 5 mg/kg×3 doses weekly in SCID mice bearing non-small cell carcinoma (NSCLC) xenografts.

FIG. 14 shows PDT response and antitumor activity of combination therapy of HPPH+PDT+Cisplatin 5 mg/kg weekly×3 dose (1 hour post PDT) in SCID mice bearing NSCLC lung cancer xenografts.

FIG. 15 shows PDT response and antitumor activity of combination therapy of HPPH+PDT+Doxorubicin 5 mg/kg×3 doses weekly in SCID mice bearing non-small cell carcinoma (NSCLC) xenografts.

FIG. 16 shows PDT response and antitumor activity of combination therapy of HPPH+PDT+Doxorubicin 5 mg/kg weekly×3 dose (1 hour post PDT) in SCID mice bearing NSCLC lung cancer xenografts.

FIG. 17 shows PDT response and antitumor activity of combination therapy of HPPH+PDT+Irinotecan 100 mg/kg weekly×4 dose (1 hour post PDT) in SCID mice bearing FaDu head and neck xenografts.

FIG. 18 shows PDT response and antitumor activity of combination therapy of HPPH+PDT+Irinotecan 100 mg/kg weekly×4 dose (1 hour post PDT) in SCID mice bearing FaDu head and neck xenografts.

FIG. 19 shows antitumor activity of doxorubicin 5 mg/kg weekly×3 doses in SCID mice bearing 85-1 head and neck xenografts.

FIG. 20 shows antitumor activity of irinotecan 100 mg/kg weekly×4 doses in SCID mice bearing 85-1 head and neck xenografts.

FIG. 21 shows antitumor activity of irinotecan 100 mg/kg weekly×4 doses in SCID mice bearing FaDu head and neck xenografts.

FIG. 22 shows antitumor activity of doxorubicin 5 mg/kg weekly×3 doses in SCID mice bearing FaDu head and neck xenografts.

FIG. 23 shows antitumor activity of doxorubicin 5 mg/kg weekly×3 doses in Balbc mice bearing colon 26 tumors.

FIG. 24 shows antitumor activity of cisplatin 5 mg/kg weekly×3 doses in BALB/c mice bearing colon 26 tumors.

FIG. 25 shows antitumor activity of doxorubicin 5 mg/kg weekly×3 doses in SCID mice bearing NSCLC 148070 lung cancer xenografts.

FIG. 26 shows antitumor activity of cisplatin 5 mg/kg weekly×3 doses in SCID mice bearing NSCLC 148070 lung cancer xenografts.

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, and method step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The present disclosure provides methods of treatment based on the use of a combination of NIR photosensitizer(s) and chemotherapy agent(s). The present disclosure also provides kits comprising NIR photosensitizer(s) and chemotherapy agent(s), and instructions for use of the NIR photosensitizer(s) and chemotherapy agent(s) (e.g., use in methods of the present disclosure.

In an aspect, the present disclosure provides methods of treatment. The methods are based on the use of a combination of NIR photosensitizer(s) and chemotherapy agent(s). The methods can be used to treat cancer in an individual. The methods can be referred to as combination treatments.

In various examples, the present disclosure describes a unexpected enhancement of long-term cure of mice bearing various types of tumors by using either HPPH [3-(1′-hexyloxy)ethyl-3-devinylpyropheopphorbide-a, 665 nm] or Photobac [3-(1′-butyloxy)ethyl-3-deacetyl-bacteriopurpurin-18-N-butyl-imide methyl ester, 787 nm] as photosensitizer in combination with clinically approved chemotherapy agents (cisplatin, doxorubicin or erlotinib). In this combination therapy approach the chemotherapy dose was much lower than the standard dose (chemo alone), which is advantageous because it would significantly reduce severe chemo-toxicity in the patients and improve their quality of life with prolong survival or cure.

In certain cases, combination therapy (PDT+chemotherapy) may reduce symptoms and prolong the life of patients significantly. This approach can be useful in treating patients with advanced cancers that are not suitable for surgery radiation therapy (e.g., patients with small cell lung cancer, bladder cancer, brain cancer, head & neck cancer esophageal cancer that cannot be completely removed by surgery).

For a successful outcome of combination therapy using porphyrin-based compound it is important that the PDT agent are highly effective (e.g., it is desirable that PDT agent does not show any skin or organ toxicity, exhibits long-wavelength absorption near 660-800 nm, produce singlet oxygen and show significant shift between the long-wavelength absorption and fluorescence, which will help in guiding the photodynamic treatment during the light exposure by fluorescence imaging). Long wavelength photosensitizers, due to deeper tissue penetration of light at NIR range can also help to treat large (less number of optical fibers can be used, which can make PDT more economical) and deeply seated tumors.

In an example, a method for treating an individual in need of treatment for cancer comprises: administering to the individual one or more NIR photosensitizer comprising a tetrapyrrolic core or reduced tetrapyrrolic core (e.g., an effective amount of one or more such NIR photosensitizer); administering of one or more chemotherapy agent (e.g., an effective amount or a sub-therapeutic amount of one or more chemotherapy agent); and irradiating the individual with electromagnetic radiation having a wavelength of 650 nm to 800 nm. In an example, the amount of chemotherapy agent is effective to reduce tumor size without significant toxicity. In an example, an effective amount of one or more NIR photosensitizer and an effective amount of one or more chemotherapy agent are administered to the individual. In another example, an effective amount of one or more NIR photosensitizer and a sub-therapeutic amount of one or more chemotherapy agent are administered to the individual. A method may further comprise one or more additional NIR photosensitizer administrations and/or one or more additional chemotherapy agent administrations and/or one or more additional irradiations.

Various NIR photosensitizers having a tetrapyrrolic core or reduced tetrapyrrolic core can be used. NIR photosensitizers have absorbance in the wavelength range of 650 nm to 800 nm, including all integer nm wavelengths and ranges therebetween. In an example, a NIR photosensitizer has an extinction coefficient or molar extinction coefficient of 30,000 or more at one or more wavelength in the range of 650 nm to 800 nm. Extinction coefficient and molar extinction coefficient can be determined by methods known in the art. Combinations of NIR photosensitizers can be used. The NIR photosensitizers can be used as therapeutic agents (e.g., PDT agents) and, optionally, as imaging (e.g., fluorescence imaging) agents. Non-limiting examples of NIR photosensitizers include HPPH 3-(1′-hexyloxy)ethyl-3-devinylpyropheopphorbide-a, Photobac 3-(1′-butyloxy)ethyl-3-deacetyl-bacteriopurpurin-18-N-butyl-imide methyl ester, and derivatives/analogs thereof. Examples of suitable NIR photosensitizers are known in the art. Examples of NIR photosensitizers include pharmaceutically acceptable derivatives and prodrugs of NIR photosensitizers known in the art. Non-limiting examples of NIR photosensitizers are described in U.S. Pat. Nos. 5,198,460, 5,314,905, and U.S. Pat. No. 5,459,159, the disclosures of which with regard to photosensitizers are incorporated herein by reference.

Various chemotherapy agents (e.g., chemotherapy drugs) can be used. Any FDA approved chemotherapy agents (e.g., chemotherapy drugs) can be used. Combinations of chemotherapy agents can be used. Non-limiting examples of chemotherapy agents and combinations include abemaciclib, abiraterone acetate, ABITREXATE® (methotrexate), ABRAXANE® (Paclitaxel albumin-stabilized nanoparticle formulation), ABVD (doxorubicin, bleomycin, vinblastine, and dacarbazine), ABVE (doxorubicin, bleomycin, vincristine sulfate, etoposide phosphate), ABVE-PC (doxorubicin, bleomycin, vincristine sulfate, etoposide phosphate, prednisone, cyclophosphamide), AC (doxorubicin and cyclophosphamide), acalabrutinib, AC-T (doxorubicin, cyclophosphamide, paclitaxel), Adcetris® (brentuximab vedotin), ADE (cytarabine, daunorubicin, etoposide), ado-trastuzumab emtansine, ADRIAMYCIN® (doxorubicin hydrochloride), afatinib dimaleate, AFINITOR® (everolimus), AKYNZEO® (netupitant and palonosetron hydrochloride), ALDARA® (imiquimod), aldesleukin, ALECENSA® (alectinib), alectinib, alemtuzumab, ALIMTA® (pemetrexed disodium), ALIQOPA® (copanlisib hydrochloride), ALKERAN® for injection (melphalan hydrochloride), ALKERAN® tablets (melphalan), ALOXI® (palonosetron hydrochloride), ALUNBRIG™ (brigatinib), ambochlorin (chlorambucil), amboclorin (chlorambucil), amifostine, aminolevulinic acid, anastrozole, aprepitant, AREDIA® (pamidronate disodium), ARIMIDEX® (anastrozole), AROMASIN® (exemestane), ARRANON® (nelarabine), arsenic trioxide, ARZERRA® (ofatumumab), asparaginase Erwinia chrysanthemi, atezolizumab, AVASTIN® (bevacizumab), avelumab, axicabtagene ciloleucel, axitinib, azacitidine, BAVENCIO® (avelumab), BEACOPP (bleomycin, etoposide, doxorubicin, cyclophosphamide, vincristine, procarbazine, prednisone), Becenum® (carmustine), Beleodaq® (belinostat), belinostat, bendamustine hydrochloride, BEP (bleomycin, etoposide, cisplatin), BESPONSA™ (inotuzumab ozogamicin), bevacizumab, bexarotene, BEXXAR® (tositumomab and iodine ¹³¹I tositumomab), bicalutamide, BICNU® (carmustine), bleomycin, blinatumomab, BLINCYTO® (blinatumomab), bortezomib, Bosulif® (bosutinib), bosutinib, brentuximab vedotin, brigatinib, BuMel (busulfan, melphalan hydrochloride), busulfan, BUSULFEX® (busulfan), cabazitaxel, CABOMETYX™ (cabozantinib-S-malate), cabozantinib-S-malate, CAF (cyclophosphamide, doxorubicin, 5-fluorouracil), CALQUENCE® (acalabrutinib), CAMPATH® (alemtuzumab), CAMPTOSAR® (irinotecan hydrochloride), capecitabine, CAPDX, CARAC™ (fluorouracil-topical), carboplatin, carboplatin-TAXOL®, carfilzomib, carmubris (carmustine), carmustine, carmustine implant, CASODEX® (bicalutamide), CEM (carboplatin, etoposide, melphalan), ceritinib, CERUBIDINE® (daunorubicin hydrochloride), cetuximab, CEV (carboplatin, etoposide phosphate, vincristine sulfate), chlorambucil, chlorambucil-prednisone, CHOP (cyclophosphamide, doxorubicin, vincristine, prednisone), cisplatin, cladribine, CLAFEN® (cyclophosphamide), clofarabine, CLOFAREX® (clofarabine), CLOLAR® (clofarabine), CMF (cyclophosphamide, methotrexate, fluorouracil), cobimetinib, COMETRIQ® (cabozantinib-S-malate), copanlisib hydrochloride, COPDAC (cyclophosphamide, vincristine sulfate, prednisone, dacarbazine), COPP (cyclophosphamide, vincristine, procarbazine, prednisone), COPP-ABV (cyclophosphamide, vincristine, procarbazine, prednisone, doxorubicin, bleomycin, vinblastine sulfate), COSMEGEN® (dactinomycin), COTELLIC® (cobimetinib), crizotinib, CVP (cyclophosphamide, vincristine, prednisolone), cyclophosphamide, CYFOS® (ifosfamide), CYRAMZA® (ramucirumab), cytarabine, cytarabine liposome, CYTOSAR-U® (cytarabine), CYTOXAN® (cyclophosphamide), dabrafenib, dacarbazine, DACOGEN® (decitabine), dactinomycin, daratumumab, DARZALEX® (daratumumab), dasatinib, daunorubicin hydrochloride, daunorubicin hydrochloride and cytarabine liposome, decitabine, defibrotide sodium, DEFITELIO® (defibrotide sodium), degarelix, denileukin diftitox, denosumab, DEPOCYT® (cytarabine liposome), dexamethasone, dexrazoxane hydrochloride, dinutuximab, docetaxel, DOXIL® (doxorubicin hydrochloride liposome), doxorubicin, doxorubicin hydrochloride, doxorubicin hydrochloride liposome, DOX-SL® (doxorubicin hydrochloride liposome), DTIC-DOME® (dacarbazine), durvalumab, EFUDEX® (fluorouracil-topical), ELITEK® (rasburicase), ELLENCE® (epirubicin hydrochloride), elotuzumab, ELOXATIN® (oxaliplatin), eltrombopag olamine, EMEND® (aprepitant), EMPLICITI® (elotuzumab), enasidenib mesylate, enzalutamide, epirubicin hydrochloride, EPOCH (etoposide, prednisone, vincristine, cyclophosphamide, and doxorubicin hydrochloride), ERBITUX® (cetuximab), eribulin mesylate, ERIVEDGE® (vismodegib), erlotinib hydrochloride, ERWINAZE® (asparaginase Erwinia chrysanthemi), ETHYOL® (amifostine), ETOPOPHOS® (etoposide phosphate), etoposide, etoposide phosphate, EVACET® (doxorubicin hydrochloride liposome), everolimus, EVISTA® (raloxifene hydrochloride), EVOMELA® (melphalan hydrochloride), exemestane, 5-FU (fluorouracil injection), 5-FU (fluorouracil-topical), FARESTON® (toremifene), FARYDAK® (panobinostat), FASLODEX® (fulvestrant), FEC (5-fluorouracil, epirubicin, cyclophosphamide), FEMARA® (letrozole), filgrastim, FLUDARA® (fludarabine phosphate), fludarabine phosphate, FLUOROPLEX® (fluorouracil-topical), fluorouracil injection, fluorouracil-topical, flutamide, FOLEX® (methotrexate), FOLEX PFS® (methotrexate), FOLFIRI (leucovorin calcium, fluorouracil, irinotecan hydrochloride), FOLFIRI-bevacizumab, FOLFIRI-cetuximab, FOLFIRINOX (leucovorin calcium, fluorouracil, irinotecan hydrochloride, oxaliplatin), FOLFOX (leucovorin calcium, fluorouracil, oxaliplatin), FOLOTYN® (pralatrexate), FU-LV (fluorouracil, leucovorin calcium), fulvestrant, Gazyva® (obinutuzumab), gefitinib, gemcitabine hydrochloride, gemcitabine-cisplatin, gemcitabine-oxaliplatin, gemtuzumab ozogamicin, GEMZAR® (gemcitabine hydrochloride), GILOTRIF® (afatinib dimaleate), GLEEVEC® (imatinib mesylate), GLIADEL® (carmustine implant), GLIADEL® wafer (carmustine implant), glucarpidase, goserelin acetate, HALAVEN® (eribulin mesylate), HEMANGEOL® (propranolol hydrochloride), HERCEPTIN® (trastuzumab), Hycamtin® (topotecan hydrochloride), HYDREA® (hydroxyurea), hydroxyurea, Hyper-CVAD (course A: cyclophosphamide, vincristine, doxorubicin, dexamethasone, cytarabine, mesna, methotrexate; and course B: methotrexate, leucovorin, sodium bicarbonate, cytarabine), IBRANCE® (palbociclib), ibritumomab tiuxetan, ibrutinib, ICE (ifosfamide, mesna, carboplatin, etoposide), ICLUSIG® (ponatinib hydrochloride), IDAMYCIN® (idarubicin hydrochloride), idarubicin hydrochloride, idelalisib, IDHIFA® (enasidenib mesylate), IFEX® (ifosfamide), ifosfamide, IFOSFAMIDUM™ (ifosfamide), imatinib mesylate, IMBRUVICA® (ibrutinib), IMFINZI® (durvalumab), imiquimod, IMLYGIC® (talimogene laherparepvec), INLYTA® (axitinib), inotuzumab ozogamicin, interferon alfa-2b, Interleukin-2 (Aldesleukin), INTRON A™ (recombinant interferon alfa-2b), iodine I 131 tositumomab and tositumomab, ipilimumab, IRESSA® (gefitinib), irinotecan, irinotecan hydrochloride, irinotecan hydrochloride liposome, ISTODAX® (romidepsin), ixabepilone, ixazomib citrate, IXEMPRA® (ixabepilone), JAKAFI® (ruxolitinib phosphate), JEB (carboplatin, etoposide phosphate, bleomycin), JEVTANA® (cabazitaxel), KADCYLA® (ado-trastuzumab emtansine), KEOXIFENE™ (raloxifene hydrochloride), KEPIVANCE® (palifermin), KEYTRUDA® (pembrolizumab), KISQALI® (ribociclib), KYMRIAH™ (ti sagenlecleucel), KYPROLIS® (carfilzomib), lanreotide acetate, lapatinib ditosylate, LARTRUVO™ (olaratumab), lenalidomide, lenvatinib mesylate, LENVIMA® (lenvatinib mesylate), letrozole, leucovorin calcium, LEUKERAN® (chlorambucil), leuprolide acetate, LEUSTATIN® (cladribine), LEVULAN® (aminolevulinic acid), LINFOLIZIN™ (chlorambucil), LIPODOX® (doxorubicin hydrochloride liposome), lomustine, LONSURF® (trifluridine and tipiracil hydrochloride), LUPRON® (leuprolide acetate), LUPRON DEPOT® (leuprolide acetate), LUPRON DEPOT-PED® (leuprolide acetate), LYNPARZA® (olaparib), MARQIBO® (vincristine sulfate liposome), MATULANE® (procarbazine hydrochloride), mechlorethamine hydrochloride, megestrol acetate, MEKINIST® (trametinib), melphalan, melphalan hydrochloride, mercaptopurine, mesna, MESNEX® (Mesna), METHAZOLASTONE™ (temozolomide), methotrexate, METHOTREXATE LPF™ (methotrexate), methylnaltrexone bromide, MEXATE® (methotrexate), MEXATE-AQ™ (methotrexate), midostaurin, mitomycin C, mitoxantrone hydrochloride, MITOZYTREX™ (mitomycin C), MOPP (mustargen, vincristine, procarbazine, prednisone), MOZOBIL™ (plerixafor), MUSTARGEN® (mechlorethamine hydrochloride), MUTAMYCIN™ (mitomycin C), MYLERAN® (busulfan), MYLOSAR® (azacitidine), MYLOTARG™ (gemtuzumab ozogamicin), nanoparticle paclitaxel (paclitaxel albumin-stabilized nanoparticle formulation), NAVELBINE® (vinorelbine tartrate), necitumumab, nelarabine, NEOSAR® (cyclophosphamide), neratinib maleate, NERLYNX® (neratinib maleate), netupitant and palonosetron hydrochloride, NEULASTA® (pegfilgrastim), NEUPOGEN® (filgrastim), NEXAVAR® (sorafenib tosylate), NILANDRON® (nilutamide), nilotinib, nilutamide, NINLARO® (ixazomib citrate), niraparib tosylate monohydrate, nivolumab, NOLVADEX® (tamoxifen citrate), NPLATE® (romiplostim), obinutuzumab, ODOMZO® (sonidegib), OEPA (vincristine sulfate, etoposide phosphate, prednisone, doxorubicin hydrochloride), ofatumumab, OFF (oxaliplatin, fluorouracil, leucovorin), olaparib, olaratumab, omacetaxine mepesuccinate, ONCASPAR® (pegaspargase), ondansetron hydrochloride, ONIVYDE® (irinotecan hydrochloride liposome), ONTAK® (denileukin diftitox), OPDIVO® (nivolumab), OPPA (vincristine sulfate, procarbazine hydrochloride, prednisone, doxorubicin hydrochloride), osimertinib, oxaliplatin, paclitaxel, paclitaxel albumin-stabilized nanoparticle formulation, PAD (bortezomib, doxorubicin hydrochloride, dexamethasone), palbociclib, palifermin, palonosetron hydrochloride, pamidronate disodium, panitumumab, panobinostat, paraplat (carboplatin), PARAPLATIN® (carboplatin), pazopanib hydrochloride, PCV (procarbazine hydrochloride, lomustine, vincristine sulfate), PEB (cisplatin, etoposide phosphate, bleomycin), pegaspargase, pegfilgrastim, peginterferon alfa-2b, PEG-INTRON® (peginterferon alfa-2b), pembrolizumab, pemetrexed disodium, PERJETA® (pertuzumab), pertuzumab, PLATINOL® (cisplatin), PLATINOL®-AQ (cisplatin), plerixafor, pomalidomide, POMALYST® (pomalidomide), ponatinib hydrochloride, PORTRAZZA® (necitumumab), pralatrexate, prednisone, procarbazine hydrochloride, PROLEUKIN® (aldesleukin), PROLIA® (denosumab), PROMACTA® (eltrombopag olamine), propranolol hydrochloride, PROVENGE® (sipuleucel-T), PURINETHOL® (mercaptopurine), PURIXAN® (mercaptopurine), radium 223 dichloride, raloxifene hydrochloride, ramucirumab, rasburicase, R-CHOP (rituximab, cyclophosphamide, doxorubicin hydrochloride, vincristine sulfate, prednisone), R-CVP (rituximab, cyclophosphamide, vincristine sulfate, prednisone), recombinant interferon alfa-2b, regorafenib, RELISTOR® (methylnaltrexone bromide), R-EPOCH (rituximab, etoposide phosphate, prednisone, vincristine sulfate, cyclophosphamide, doxorubicin hydrochloride), REVLIMID® (lenalidomide), RHEUMATREX® (methotrexate), ribociclib, R-ICE (rituximab, ifosfamide, carboplatin, etoposide phosphate), RITUXAN® (rituximab), RITUXAN HYCELA′ (rituximab and hyaluronidase human), rituximab, rituximab and hyaluronidase human, rolapitant hydrochloride, romidepsin, romiplostim, rubidomycin (daunorubicin hydrochloride), RUBRACA® (rucaparib camsylate), rucaparib camsylate, ruxolitinib phosphate, RYDAPT® (midostaurin), SCLEROSOL® Intrapleural Aerosol (Talc), siltuximab, sipuleucel-T, SOMATULINE® Depot (lanreotide acetate), sonidegib, sorafenib tosylate, SPRYCEL® (dasatinib), Stanford V (mechlorethamine hydrochloride, doxorubicin hydrochloride, vinblastine sulfate, vincristine sulfate, bleomycin, etoposide phosphate, prednisone), sterile talc powder (Talc), STERITALC® (Talc), STIVARGA® (regorafenib), sunitinib malate, SUTENT® (sunitinib malate), SYLATRON′ (peginterferon alfa-2b), SYLVANT® (siltuximab), SYNRIBO′ (omacetaxine mepesuccinate), TABLOID® (thioguanine), TAC (docetaxel, doxorubicin hydrochloride, cyclophosphamide), TAFINLAR® (dabrafenib), TAGRISSO® (osimertinib), Talc, talimogene laherparepvec, tamoxifen citrate, TARABINE PFS® (cytarabine), TARCEVA® (erlotinib hydrochloride), TARGRETIN® (bexarotene), TASIGNA® (nilotinib), TAXOL® (Paclitaxel), TAXOTERE® (docetaxel), TECENTRIQ® (atezolizumab), TEMODAR® (temozolomide), temozolomide, temsirolimus, thalidomide, THALOMID® (thalidomide), thioguanine, thiotepa, ti sagenlecleucel, TOLAK′ (fluorouracil—topical), topotecan hydrochloride, toremifene, TORISEL® (temsirolimus), tositumomab and iodine ¹³¹I tositumomab, TOTECT® (dexrazoxane hydrochloride), TPF (docetaxel, cisplatin, fluorouracil), trabectedin, trametinib, trastuzumab, TREANDA® (bendamustine hydrochloride), trifluridine and tipiracil hydrochloride, TRISENOX® (arsenic trioxide), TYKERB® (lapatinib ditosylate), UNITUXIN′ (dinutuximab), uridine triacetate, VAC (vincristine sulfate, dactinomycin, cyclophosphamide), valrubicin, VALSTAR® (valrubicin), vandetanib, VAMP (vincristine sulfate, doxorubicin hydrochloride, methotrexate, prednisone), VARUBI® (rolapitant hydrochloride), VECTIBIX® (panitumumab), VeIP (vinblastine sulfate, ifosfamide, cisplatin), VELBAN® (vinblastine sulfate), VELCADE® (bortezomib), VELSAR® (vinblastine sulfate), vemurafenib, VENCLEXTA′ (venetoclax), venetoclax, VERZENIO′ (abemaciclib), VIADUR® (leuprolide acetate), VIDAZA® (azacitidine), vinblastine sulfate, VINCASAR PFS® (vincristine sulfate), vincristine sulfate, vincristine sulfate liposome, vinorelbine tartrate, VIP (etoposide phosphate, ifosfamide, cisplatin), vismodegib, VISTOGARD® (uridine triacetate), VORAXAZE® (glucarpidase), vorinostat, VOTRIENT® (pazopanib hydrochloride), VYXEOS' (daunorubicin hydrochloride and cytarabine liposome), WELLCOVORIN® (leucovorin calcium), XALKORI® (crizotinib), XELODA® (capecitabine), XELIRI (capecitabine, irinotecan hydrochloride), XELOX (capecitabine, oxaliplatin), XGEVA® (denosumab), XOFIGO® (radium 223 dichloride), XTANDI® (enzalutamide), YERVOY® (ipilimumab), YESCARTA′ (axicabtagene ciloleucel), YONDELIS® (trabectedin), ZALTRAP® (ziv-aflibercept), ZARXIO® (filgrastim), ZEJULA® (niraparib tosylate monohydrate), ZELBORAF® (vemurafenib), ZEVALIN® (ibritumomab tiuxetan), ZINECARD® (dexrazoxane hydrochloride), ziv-aflibercept, ZOFRAN® (ondansetron hydrochloride), ZOLADEX® (goserelin acetate), zoledronic acid, ZOLINZA® (vorinostat), ZOMETA® (zoledronic acid), ZYDELIG® (idelalisib), ZYKADIA® (ceritinib), and ZYTIGA® (abiraterone acetate).

Various amounts of the NIR photosensitizer(s) and chemotherapy agent(s) can be used. In an example, an effective amount of NIR photosensitizer(s) and an effective amount of chemotherapy agent(s) are administered. In another example, an effective amount of NIR photosensitizer(s) and a sub-therapeutic amount of chemotherapy agent(s) are administered. A sub-therapeutic amount of a chemotherapy agent is an amount (e.g., a dose or multiple doses) of the chemotherapy agent that is lower than usual or typical amount of chemotherapy agent when administered alone or in the absence of a NIR photosensitizer for treatment of cancer. In an example, a sub-therapeutic amount of a chemotherapy agent provides at least the same effect (e.g., decreased tumor volume), but with less toxicity, as an effective amount of the chemotherapy agent administered at its usual or typical therapeutic level alone or in the absence of a NIR photosensitizer.

The term “effective amount” as used herein refers to an amount of an agent or combination of agents (e.g., chemotherapy agent(s) and/or NIR photosensitizer(s)) sufficient to achieve, in a single or multiple doses or administration(s), the intended purpose or achieve a desired result of the administration. The exact amount desired or required will vary depending on the particular compound or composition used, its mode of administration, type of cancer, patient specifics, and the like. Appropriate effective amount can be determined by one of ordinary skill in the art informed by the instant disclosure using only routine experimentation.

In various examples, the effective amount of chemotherapy agent is 25% to 75% (e.g., 50%) 25% of an effective amount of chemotherapy agent necessary to provide the same effect in the absence of administration of the NIR photosensitizer. In various examples, the effective amount of chemotherapy agent is 25% or less, 40% or less, 50% or less, 60% or less, or 75% or less than effective amount of chemotherapy agent necessary to provide the same effect in the absence of administration of the NIR photosensitizer.

NIR photosensitizer(s) and/or chemotherapy agent(s) can be introduced into an individual by any suitable administration route. Suitable administration routes are known in the art. Non-limiting examples of administration include parenteral, subcutaneous, intraperitoneal, intramuscular, intravenous, intratumoral, mucosal, topical, intradermal, and oral administration. Administration can be done by way of a single dose or it can be done by multiple doses that are spaced apart. Administration can also be on a continuous basis (e.g., infusion) over a desired period of time.

The administrations and irradiation can be carried out in various ways and in various orders. Typically, administration of the NIR photosensitizer(s) is/are carried out first, and, subsequently, the chemotherapy agent(s) is/are is administered. The irradiation is carried out after administration of the NIR photosensitizer(s) and before administration of the chemotherapy agent(s) or after administration of both the NIR photosensitizer(s) and chemotherapy agent(s). In an example, the administration comprises i) administration of the NIR photosensitizer, and ii) after completion of the administration of the NIR photosensitizer and irradiation of the individual, administration of the chemotherapy agent.

In an example, the chemotherapy agent is administered (e.g., administration initiated) 30 minutes to 90 minutes, including all integer minute values and ranges therebetween, after administration (e.g., first administration) of the NIR photosensitizer(s) or after administration (e.g., first administration) of the NIR photosensitizer(s) and irradiation.

In other examples, the chemotherapy agent is administered 45 minutes to 75 minutes or 55 minutes to 65 minutes after administration of the NIR photosensitizer(s) or after administration of the NIR photosensitizer(s) and irradiation. In another example, the chemotherapy agent is administered one hour after administration of the NIR photosensitizer(s) or after administration of the NIR photosensitizer(s) and irradiation.

Without intending to be bound by any particular theory, it is considered that the irradiation causes a response (e.g., photodynamic therapy response) in the individual. Suitable irradiation protocols (e.g., PDT protocols) for NIR photosensitizers are known in the art. “Irradiating” and “irradiation” as used herein includes exposing an individual to a selected wavelength or wavelengths of light. It is desirable that the irradiating wavelength is selected to match the wavelength(s) which excite the NIR photosensitizer(s). It is desirable that the radiation wavelength(s) matches the excitation wavelength(s) of the NIR photosensitizer(s) and has low absorption by the non-target tissues of the individual, including blood proteins, because the non-target tissues have no absorbed the NIR photosensitizer(s).

Irradiation is further defined herein by its coherence (laser) or non-coherence (non-laser), as well as intensity, duration, and timing with respect to dosing using the NIR photosensitizing compound. The intensity or fluence rate must be sufficient for the light to reach the target tissue. The duration or total fluence dose must be sufficient to photoactivate enough NIR photosensitizing compound to act on the target tissue. Timing with respect to dosing with the NIR photosensitizing compound is important, because 1) the administered NIR photosensitizing compound requires some time to home in on target tissue and 2) the blood level of many NIR photosensitizing compounds decreases with time. The radiation energy is provided by an energy source, such as a laser or cold cathode light source, that is external to the individual, or that is implanted in the individual, or that is introduced into an individual, such as by a catheter, optical fiber or by ingesting the light source in capsule or pill form (e.g., as disclosed in. U.S. Pat. No. 6,273,904 (2001)).

A method of the present disclosure can be used to treat an individual with (e.g., diagnosed with) cancer. The treatment can have various results. In various examples, a method of the present disclosure results in at least one or more of the following: complete cure of the individual, remission, increased long-term survival of the individual, or reduced tumor volume for at least one tumor compared to PDT treatment alone using the same NIR photosensitizer or chemotherapy alone using the same chemotherapy agent alone.

Methods of the present disclosure can be used on various individuals. In various examples, an individual is a human or non-human mammal. Examples of non-human mammals include, but are not limited to, farm animals, such as cows, hogs, sheep, and the like, as well as pet or sport animals such as horses, dogs, cats, and the like. Additional non-limiting examples of individuals include rabbits, rats, and mice.

A method may also comprise visualization of the cancer (e.g., visualization of one or more tumors) after administration of the NIR photosensitizer. The visualization (e.g., fluorescence imaging) can be used to determine personalized treatment for an individual. For example, visualization is carried using fluorescence imaging. A method may further comprise further comprise surgical intervention (e.g., surgical removal of at least a portion of or all of a cancerous tissue from the individual). The surgical removal can be guided by the visualization (e.g., fluorescence imaging).

Methods of the present disclosure can be used to treat various cancers (e.g., a tumor or tumors related to a cancer). Non-limiting examples of cancers include lung cancer, head and/or neck cancer, esophageal cancer, laryngeal cancer, breast cancer, pancreatic cancer, renal cancer, bladder cancer, ovarian cancer, prostate cancer, testicular cancer, and combinations thereof.

In an example, the individual is in need of treatment for bladder cancer and BCG is administered after administration of both the NIR photosensitizer and the chemotherapy agent.

In an aspect, the present disclosure provides pharmaceutical compositions comprising one or more NIR photosensitizer and one or more chemotherapy agent. The compositions may comprise one or more pharmaceutically acceptable carrier. In various examples, the pharmaceutical composition further comprises Tween® 80 or Pluronic™ F-127. In an example, a pharmaceutical composition further comprises Bacillus Calmette-Guerin (BCG) vaccine.

The compositions can include one or more standard pharmaceutically acceptable carriers. The compositions can include solutions, suspensions, emulsions, and solid injectable compositions that are dissolved or suspended in a solvent before use. The injections can be prepared by dissolving, suspending or emulsifying one or more of the active ingredients in a diluent. Examples of diluents are distilled water for injection, physiological saline, vegetable oil, alcohol, and a combination thereof. Further, the injections can contain stabilizers, solubilizers, suspending agents, emulsifiers, soothing agents, buffers, preservatives, etc. The injections are sterilized in the final formulation step or prepared by sterile procedure. The pharmaceutical composition of the invention can also be formulated into a sterile solid preparation, for example, by freeze-drying, and can be used after sterilized or dissolved in sterile injectable water or other sterile diluent(s) immediately before use. Non-limiting examples of pharmaceutically acceptable carriers can be found in: Remington: The Science and Practice of Pharmacy (2005) 21st Edition, Philadelphia, Pa. Lippincott Williams & Wilkins.

In another aspect, the present disclosure provides kits. In an example, a kit comprises NIR photosensitizer(s) and chemotherapy agent(s) and instructions for their use. In another example, a kit further comprises Bacillus Calmette-Guerin (BCG) vaccine.

The kits can comprise pharmaceutical preparations containing any one or any combination of the compounds (e.g., NIR photosensitizer(s) and chemotherapy agent(s)) described herein. In an example, a kit is or includes a closed or sealed package that contains the pharmaceutical preparation. In certain examples, the package can comprise one or more closed or sealed vials, bottles, blister (bubble) packs, or any other suitable packaging for the sale, or distribution, or use of the pharmaceutical compounds and compositions comprising them. The printed material can include printed information. The printed information can be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of cancer and/or any disorder associated with cancer. In examples, the kit includes a label describing the contents of the container and providing indications and/or instructions regarding use of the contents of the kit to treat any cancer.

The steps of the methods described in the various embodiments and examples disclosed herein are sufficient to carry out the methods of the present disclosure. Thus, in various examples, a method consists essentially of a combination of the steps of the methods disclosed herein. In various other examples, a method consists of such steps.

The following examples are presented to illustrate the present disclosure. It is not intended to limiting in any matter.

### Example 1

This example provides a description of compounds of the present disclosure, and synthesis and uses of the same.

The photosensitizers; HPPH and Photobac were selected for the combination studies because both NIR agents developed in our laboratory are highly effective in various animal tumor models.

HPPH is currently undergoing Phase II clinical trials of head & neck cancer in the United States. It was also found effective for the treatment of skin cancer (basal cell carcinoma) early lung cancer and esophageal cancers (Phase I human clinical trials). All the preclinical pharmacokinetic (PK)/pharmacodynamics (PD) and toxicity studies of Photobac in rats and dogs have been completed following the US FDA requirements in a GLP facility.

For a proof-of-principle study, the initial combination therapy was performed by using HPPH and Photobac as photosensitizers for treating mice bearing lung, head & neck and bladder cancers.

(A) Lung Cancer

Lung cancer is the leading cause of cancer deaths in the United States for both women and men. Surgery remains the primary treatment modality for locoregional disease. However, local recurrence remains a significant problem despite modest improvement in survival from adjuvant chemotherapy. Adjuvant regional therapy can enhance local disease control and further improve survival. Due to the association of lung cancer with tobacco use, many patients also suffer from impaired lung function resulting from chronic obstructive pulmonary disease (COPD). Consequently, surgical resection of some early stage tumors may be contraindicated because of inadequate pulmonary reserve. Additionally, up to 10% of successfully resected or radiated patients with lung cancer subsequently develop a second primary lung neoplasm, and another operation or further radiotherapy may not be feasible at that point. Thus, therapeutic approaches that spare functional lung tissue are required by many patients in whom lung cancer is diagnosed. Photodynamic therapy (PDT) is an evolving modality that can meet a number of therapeutic challenges in lung cancer. It can be developed as an adjuvant intraoperative therapy and as alternative to resection or radiotherapy. It can also be combined with other treatment modalities.

To investigate the advantages of combination therapy in lung cancer, SCID mice bearing non-small cell carcinoma (NSCLC) xenografts (5 mice/group) were treated with (i) HPPH−PDT (drug dose: 0.47 μmol/kg, light dose: 135 J/cm², 75 mW/cm², wavelength: 665 nm at 24 h post-injection) (ii) cisplatin or doxorubicin alone 5 mg/kg×3 doses weekly×3 weeks and (iii) HPPH−PDT and doxorubicin or cisplatin after 1 h PDT treatment (first treatment) under similar treatment parameters.

To investigate the advantages of combination therapy of Photobac-PDT with doxorubicin, SCID mice bearing non-small cell carcinoma (NSCLC) xenografts (5 mice/group) were treated with (i) Photobac-PDT (drug dose: 0.50 μmol/kg, light dose: 135 J/cm², 75 mW/cm², wavelength: 787 nm at 24 h post-injection) (ii) doxorubicin alone 5 mg/kg×3 doses weekly and (iii) Photobac-PDT and doxorubicin after 1 h PDT treatment (first treatment) under similar treatment parameters.

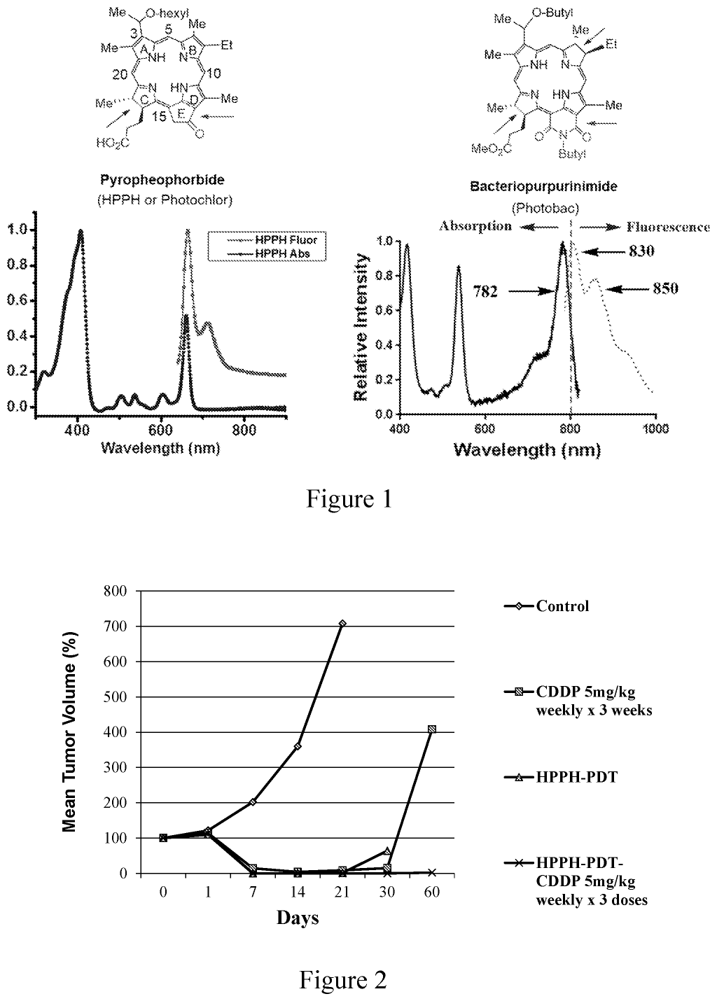

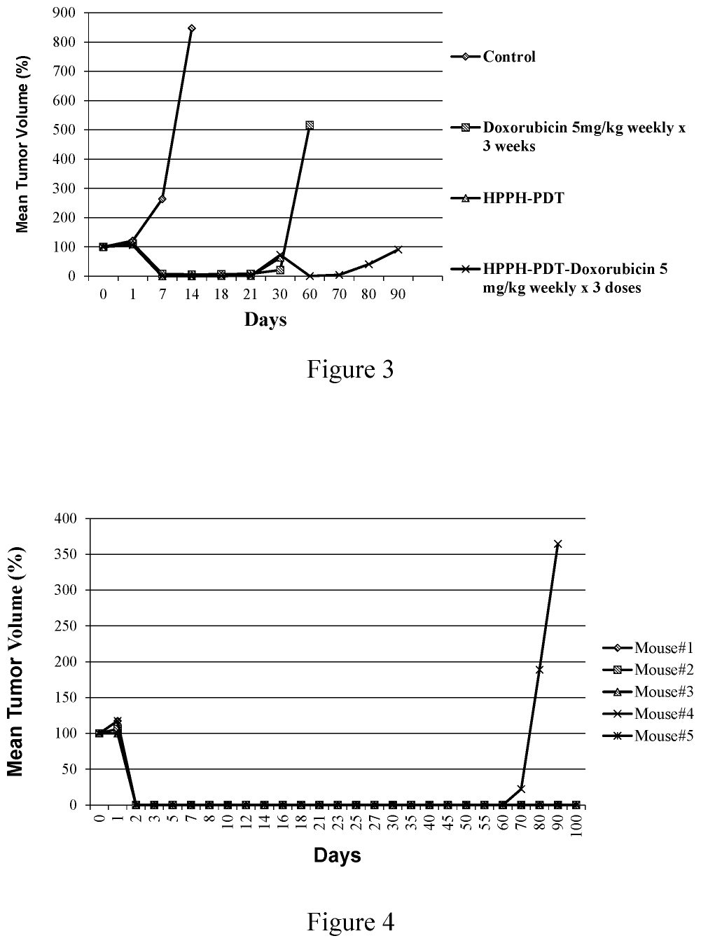

The data in FIGS. 2-4 showed the effects of antitumor activity and toxicity of HPPH−PDT±Cisplatin or Doxorubicin in SCID mice bearing NSCLC lung cancer xenografts.

The results indicate that Cisplatin or Doxorubicin (both doses of 5 mg/kg) weekly×3 doses had antitumor activity against this tumor type in combination with HPPH or Photobac with PDT and CR of 80% (8/10 mice) with both Cisplatin and Doxorubicin. The combination of PDT with HPPH had 20% CR in comparison to combination of 80% CR. The alone drug was not effective. The enhanced antitumor activity of the combination is highly significant. As far as toxicity is concerned, no significant effects were observed in the used doses.

Experimental Details

Section A: Compare the effectiveness of HPPH and Photobac in human non-small cell lung cancer xenografts in SCID mice.

Materials and Methods

### Materials:

1) Mice Strain: SCID mice (C.B Igh-1b Icr Tac Prkdc scid)
2) Tumors: Human non-small cell cancer xenografts
3) Drugs and doses:
a) HPPH 0.47 μmol/kg
b) Photobac 0.50 μmol/kg
c) Cisplatin 5 mg/kg

### Methods:

1) Drug administration: intravenous (i.v.) push
2) Drug preparation and schedules
HPPH: PDT was done 24 hours after photosensitizer (PS) treatment
Photobac: PDT was done 24 hours after PS treatment
Cisplatin: At 5 mg/kg weekly×3 doses
Doxorubicin: At 5 mg/kg weekly×3 doses
Irinotecan 100 mg/kg weekly×4 doses
   Chemotherapy: First dose 1 hour post PDT and 2 doses weekly thereafter for weekly schedule for i.v. treatments.
   3) Tumor transplantation: the xenografts used for antitumor activity were transplanted subcutaneously. The treatment was initiated 7-10 days post transplantation.
   4) Tumor measurement: two axes (mm) of tumor (L, longest axis; W, shortest axis) were measured with the aid of a Vernier caliper. Tumor weight (mg) was estimated as a formula tumor weight ½ (L×W2). Tumor measurements were taken after the scabbing healed post PDT and at least three times a week for post therapy and twice a week therefore.
   5) Tumor regression: complete tumor regression (CR) was defined as the inability to detect tumor by palpation at the initial site of tumor appearance for more than two months post therapy. Thumor regrowth after CR occurred was observed in less 5% of mice. Partial tumor regression (PR) was defined as ≥50% reduction in initial tumor size. Normally, 5 mice per treatment group were included in these experiment groups and repeated at least twice.

PDT of Subcutaneous Tumors—Tumor bearing mice are injected via the tail vein with HPPH or derivatives at doses that are non-toxic unless exposed to light. For tail vein injections, mice were gently restrained in approved holders and their tails were briefly (less than 1 minute) dipped in warm sterile water (˜40° C.). Photosensitizers are injected in a volume of less than 0.2 mL into the tail vein using a 27-gauge needle.

The photosensitizers, at 24 hours (or the optimal time determined by optical imaging) after administration of HPPH or derivatives, the animals were partially restrained allowing leg movement, in specially designed holders without anesthesia. The animal restraint procedure has been demonstrated to and approved by Laboratory of animal resources (LAR).

Tumors are exposed to visible light (1 cm diameter) at power densities of less than 100 mW/cm². The light sources are either a dye laser (with a tunable range of 600 to 800 nm), at the optimal excitation wavelength for the individual photosensitizer; or a pulsed laser with a range of 460 to 800 nm. The treatment will vary, depending on the light dose (J/cm²) and fluence rate (mW/cm²) required, but usually is for approximately 30 minutes. The mice are held still in the specially designed holders with no problems during treatment.

After light exposure, the mice are monitored closely for at least 1 hour and then daily until the re-growing tumors reach no more than 2 cm in the greatest dimension or for a maximum of 60 days (post treatment) at which time they are euthanized. All animals will be euthanized within 90 days of tumor implantation.

NSCLC (Lung cancer): NSCLC (Squamous cell carcinoma of the floor of mouth head and neck carcinoma).

Previous published data with PDT in lung cancer tumors have shown increase in response rates and want to see if the response rates are better in combination with chemotherapy.

(B) Head and Neck Cancer

Head and neck squamous cell carcinoma (HNSCC) is the most common cancer occurring in men. Median survival of patients with late stage, recurrent and metastatic head and neck cancer is less than a year. Thus there is a need of an effective treatment. Recurrent head and neck cancer is always a major problem. In some cases after major surgery with chemotherapy and/or radiotherapy it is not possible to gain access to the recurrent cancer side. The advantage of PDT is that the patients after having the light treatment, if necessary, can undergo other treatment modality, e.g. chemotherapy. PDT can be done before or after the treatment.

HPPH, one of the photosensitizers developed in our laboratory, is currently being used for the treatment of head and neck cancer in RPCI. The results are impressive in patients with loco-regional tumors. However, this approach is not beneficial in treating patients where the tumor has already been metastasized. Besides, in some of the patient population (5 to 10%) the cancer regrowth was also observed. Therefore, a combination therapy with chemotherapy (before or after PDT) at a lower dose could be extremely useful to cancer patients.

To investigate the advantages of combination therapy in head & neck cancer, SCID mice bearing FaDu xenografts (5 mice/group) were treated with (i) HPPH−PDT (drug dose: 0.47 μmol/kg, light dose: 135 J/cm², 75 mW/cm², wavelength: 665 nm at 24 h post-injection) (ii) Irinotecan alone 100 mg/kg weekly×4 doses weekly and (iii) HPPH−PDT and irinotecan 100 mg/kg weekly/4 doses. First dose of irinotecan: 1 h post-PDT treatment.

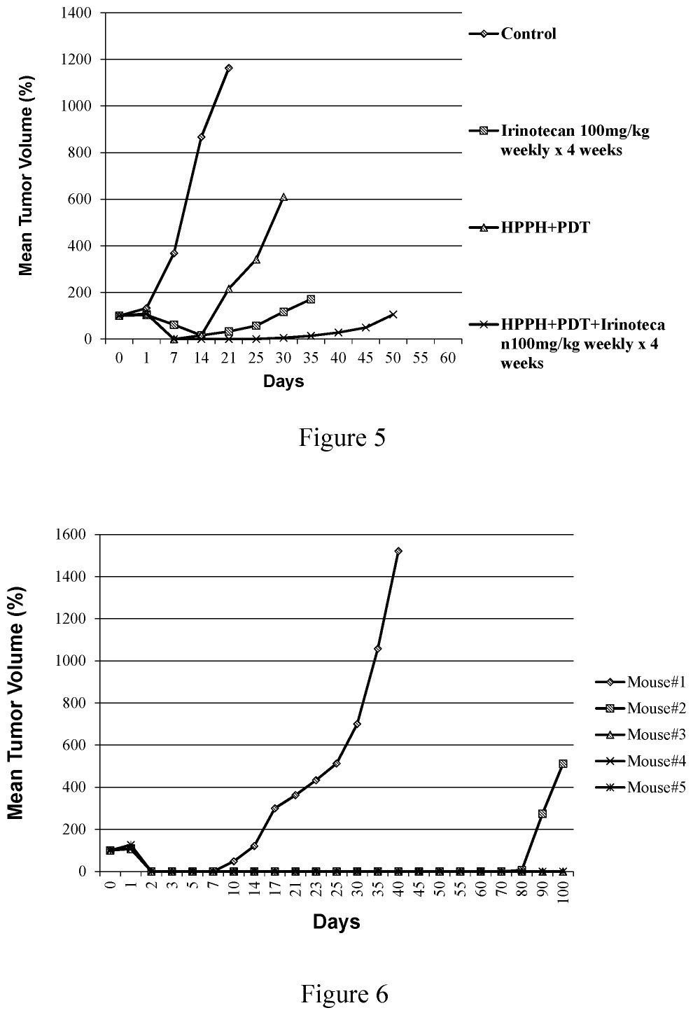

The data in FIGS. 5 and 6 showed the effects of antitumor activity and toxicity of HPPH±PDT±Irinotecan or Doxorubicin in SCID mice bearing FaDu head and neck cancer xenografts.

The results indicate that Irinotecan at 100 mg/kg weekly×4 doses had antitumor activity against this tumor type in combination with HPPH and PDT and CR of 73% (11/15 mice) and in combination with Photobac 60% CR (3/5 mice). The alone drug was not effective. The enhanced antitumor activity of the combination is highly significant. As for as toxicity is concerned, no significant effects were observed in the used doses.

Experimental Details

Section B: Compare the Effectiveness of HPPH and Photobac in Head and Neck Cancer Xenografts in SCID Mice.

### Materials and Methods:

### A. Materials

### 1) Mice Straing SCID Mice (C.B Igh-1b Icr Tac Prkdc Scid)

Human head and neck cancer xenografts (FaDu and H&N 85-1)

### 2) Drugs and Doses:

a) HPPH 0.47 μmol/kg
b) Photobac 0.50 μmol/kg
c) Doxorubicin 5 mg/kg
d) Irinotecan 100 mg/kg

### B. Methods

1) Route of drug administration: intravenous (i.v.) push
2) Drug preparation and schedules
HPPH: PDT was done 24 hours after PS treatment
Photobac: PDT was done 24 hours after PS treatment
Doxorubicin: At 5 mg/kg weekly×3 doses
Irinotecan: 100 mg/kg weekly×4 doses
   Chemotherapy: first dose 1 hour post PDT and 2 doses weekly thereafter for doxorubicin and 3 doses for irinotecan for weekly schedule for i.v. treatments.
   3) Tumor Transplantation: the xenografts used for antitumor activity were transplanted subcutaneously. The treatment was initiated 7-10 post transplantation.
   4) Tumor measurement: two axes (mm) of tumor (L, longest axis; W, shortest axis) were measured with the aid of a Vernier caliper. Tumor weight (mg) was estimated as a formula tumor weight=½ (L×W2). Tumor measurements were taken after the scabbing healed post PDT and at least three times a week for post therapy and twice a week therefore.
   5) Tumor regression: complete tumor regression (CR) was defined as the inability to detect tumor by palpation at the initial site of tumor appearance for more than two months post therapy. Tumor regrowth after CR occurred was observed in less 5% of mice. Partial tumor regression (PR) was defined as ≥50% reduction in initial tumor size. Normally, 5 mice per treatment group were included in these experiment groups.

PDT of Subcutaneous Tumors—Tumor bearing mice are injected via the tail vein with HPPH or derivatives at doses that are non-toxic unless exposed to light. For tail vein injections, mice are gently restrained in approved holders and their tails are briefly (less than 1 minute) dipped in warm sterile water (˜40° C.). Photosensitizers are injected in a volume of less than 0.2 mL into the tail vein using a 27-gauge needle.

The photosensitizers, at 24 hours (or the optimal time determined by optical imaging) after administration of HPPH or derivatives, the animals were partially restrained allowing leg movement, in specially designed holders without anesthesia. The animal restraint procedure has been demonstrated to and approved by Laboratory of animal resources (LAR).

Tumors are exposed to visible light (1 cm diameter) at power densities of less than 100 mW/cm². The light sources are either a dye laser (with a tunable range of 600 to 800 nm), at the optimal excitation wavelength for the individual photosensitizer; or a pulsed laser with a range of 460 to 800 nm. The treatment will vary, depending on the light dose (J/cm²) and fluence rate (mW/cm²) required, but usually is for approximately 30 minutes. The mice are held still in the specially designed holders with no problems during treatment.

After light exposure, the mice are monitored closely for at least 1 hour and then daily until the re-growing tumors reach no more than 2 cm in the greatest dimension or for a maximum of 60 days (post treatment) at which time they are euthanized. All animals will be euthanized within 90 days of tumor implantation.

FaDu (Head and neck). Previous data with PDT in head and neck cancer tumors have shown response rates and we wanted to see if the response rates are better in combination with chemotherapy in this tumor type.

(C) Urinary Bladder Cancer

Bladder cancer is the commonest malignancy of the urinary tract, with the incidence being four times higher in men than in women. Approximately 75 to 85% of patients will have disease confined to the mucosa (Ta) or submucosa (T1), that is, non-muscle invasive bladder cancer (NMIBC), which was previously known as ‘superficial’ bladder cancer. NMIBC requires adjuvant intravesical chemotherapy and/or immunotherapy (BCG). Porphyrin-based compounds (e. G., Photofrin), 5-aminolevulenic acid (5-ALA), a prodrug for the photosensitizer protoporphyrin-IX have been used in diagnosis of cancer by fluorescence and treatment by PDT. However, both these compounds exhibit weak absorption at its long wavelength absorption at 630 nm, which limits its light penetration. Photofrin is an effective drug, put patients suffers with severe skin phototoxicity, and patients are advised to be away from sunlight at least 6-8 weeks after the light treatment.

Fortunately, the effective photosensitizers HPPH and Photobac discovered in our laboratory exhibit long wavelength absorption and fluorescence at near infrared region (HPPH: 665 nm and Photobac: 787 nm), which could help in cancer diagnosis by fluorescence and treatment by PDT for both superficial and deeply seated cancer. The combination approach of HPPH−PDT with BCG should further enhance the long-term tumor cure.

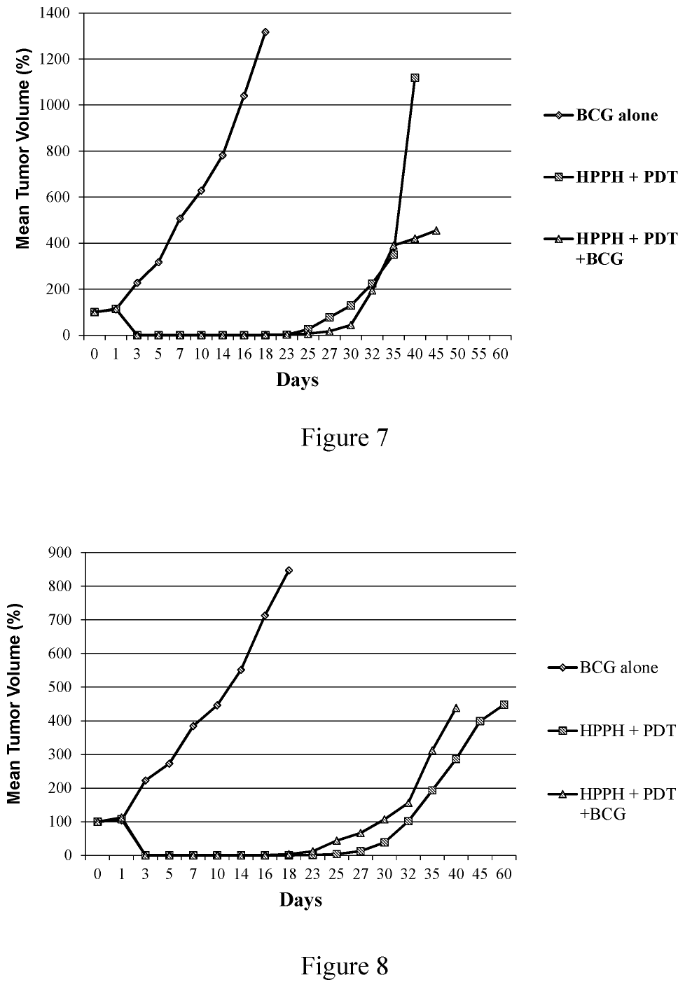

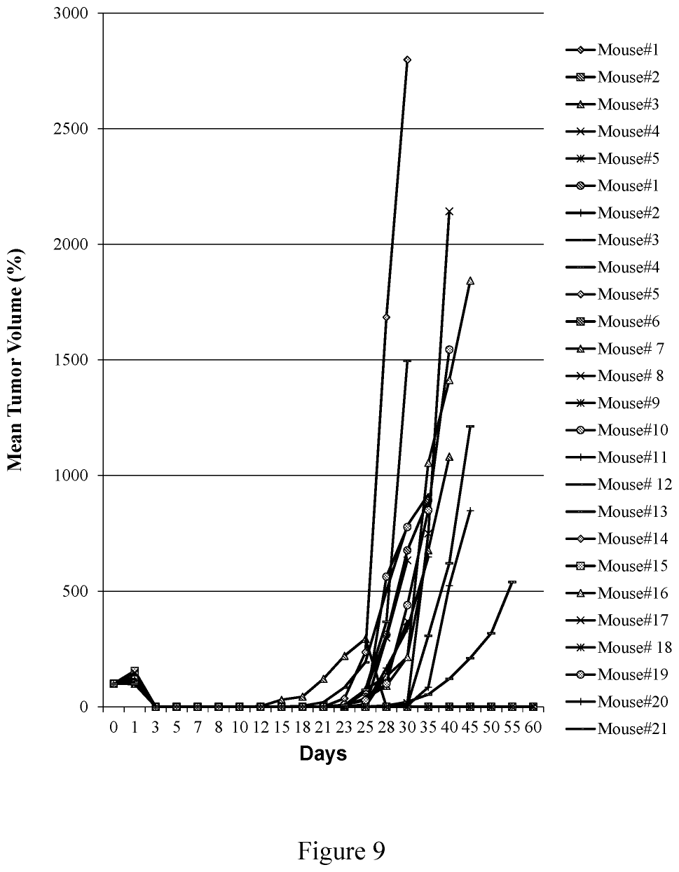

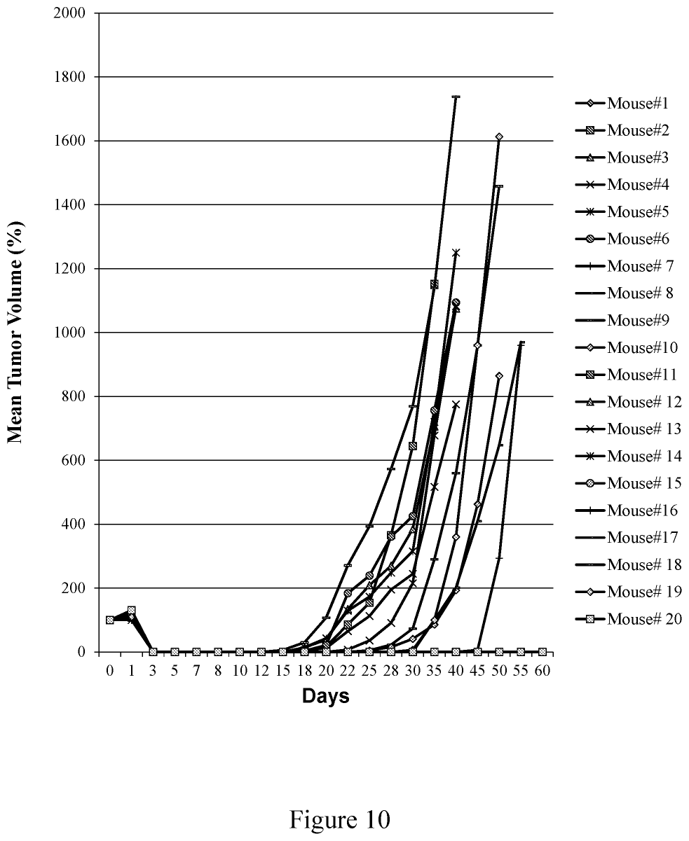

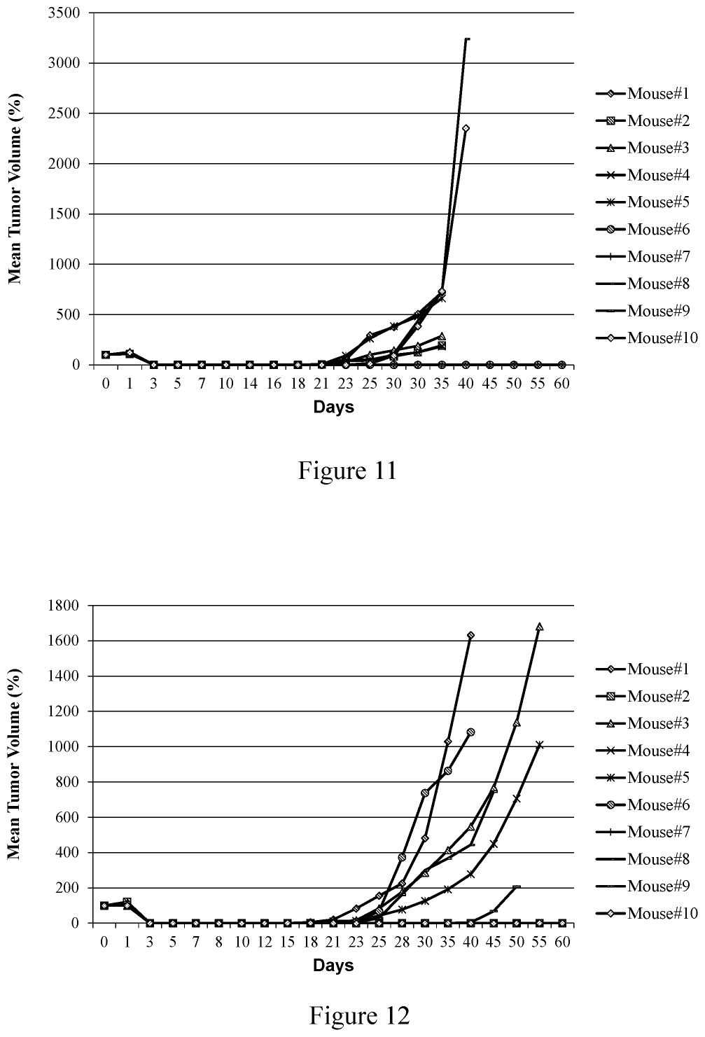

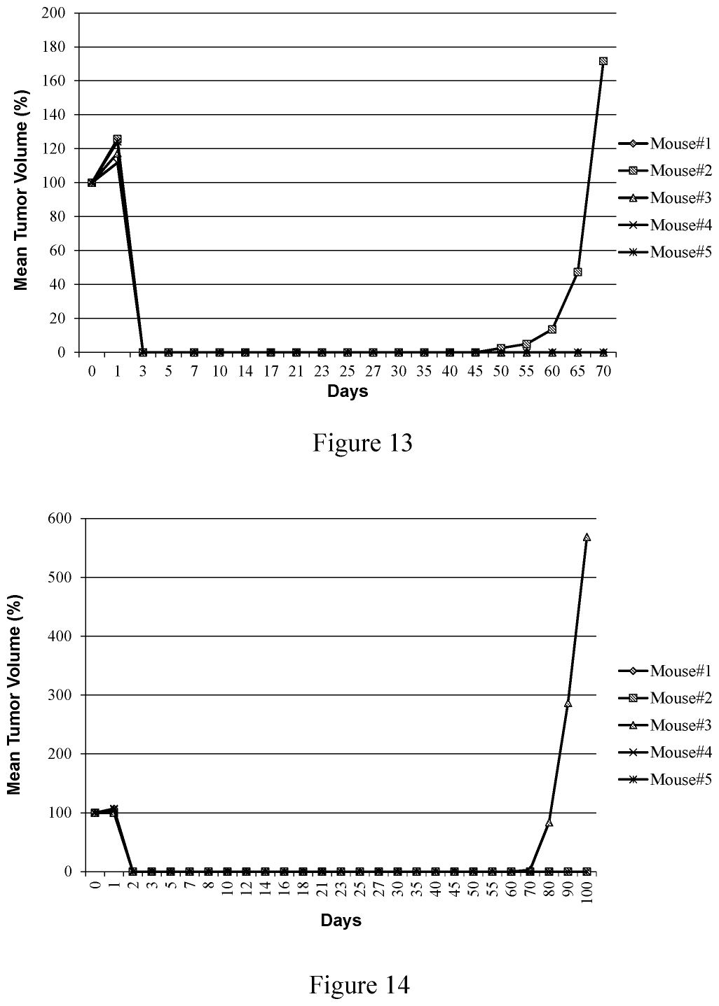

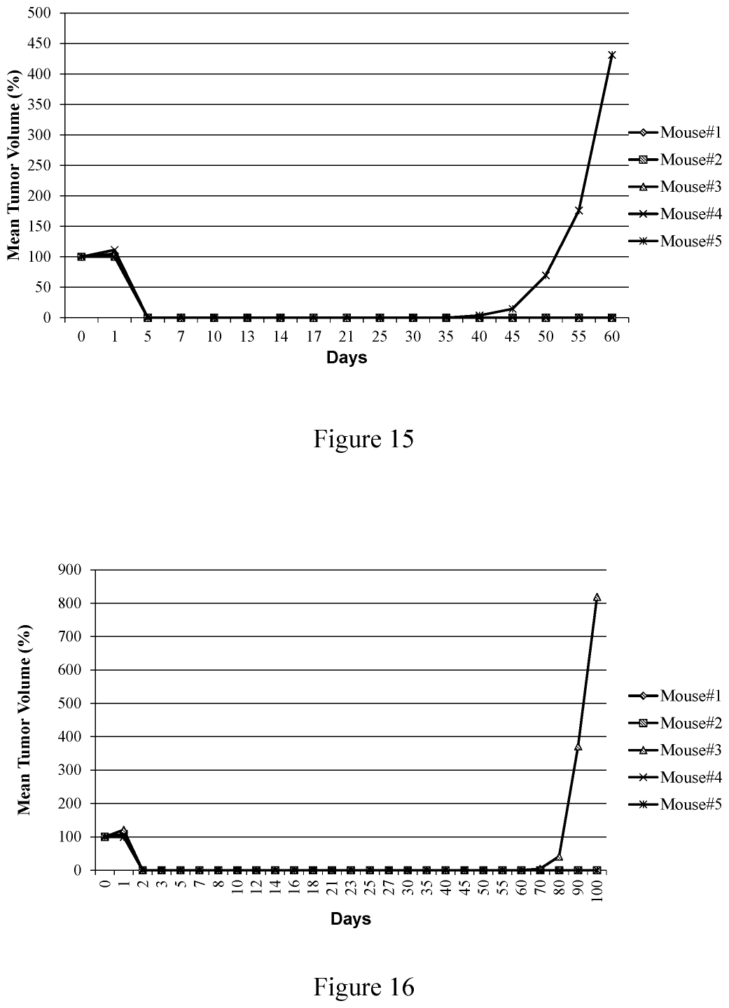

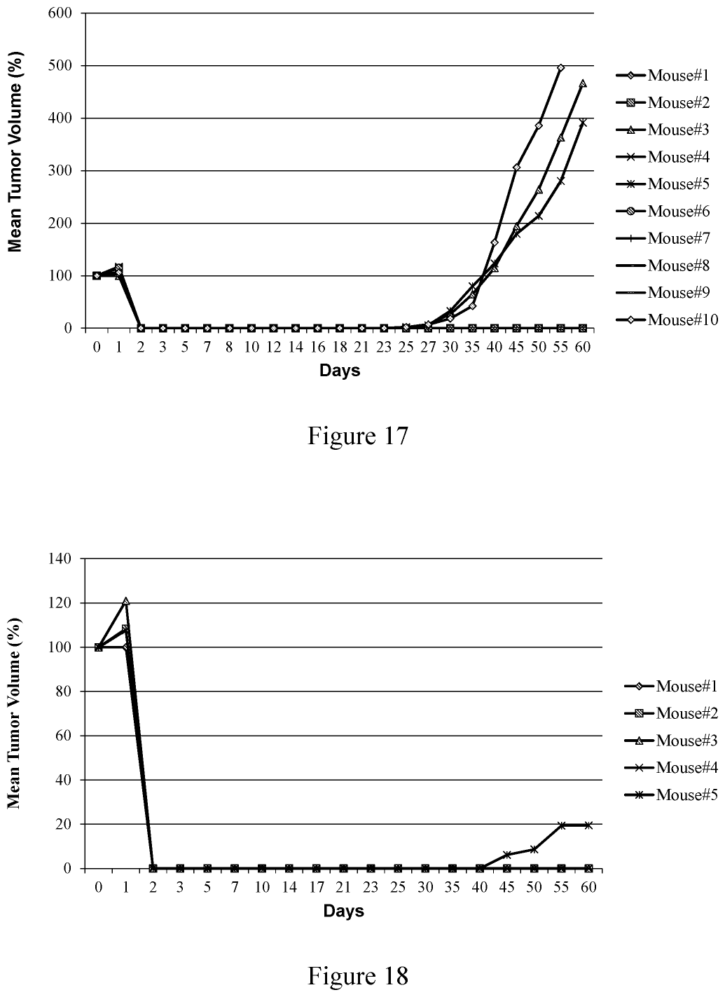

The data in FIGS. 7 and 8 showed the effects of antitumor activity and toxicity of HPPH±PDT±BCG in SCID mice bearing urinary bladder cancer xenografts.

The results indicate that BCG at 2×10⁶ cells in 100 μl subcutaneously (SC) weekly×3 doses had antitumor activity against this tumor type (UMUC-3 and T24 xenografts) in combination with HPPH and PDT with CR of 32% vs 40% in UMUC-3 and T24 respectively. The alone drug was not effective. The enhanced antitumor activity of the combination is significant. As for toxicity is concerned, no significant effects were observed in the used doses.

Experimental Details

Section C: Compare the Effectiveness of HPPH and BCG Bladder Cancer Xenografts in SCID Mice.

Materials and Methods

### A. Materials

1) Mice Strain: SCID mice (C.B Igh-1b Icr Tac Prkdc scid)
Human urinary bladder cancer xenografts (UMUC-3 and T24)
2) Drugs and doses
a) HPPH 0.47 μmol/kg
b) Immunotherapy (BCG vaccine)˜2×10⁶ cells in 100 ul/dose subcutaneously (SQ)

### B. Methods

1) Route of drug administration: intravenous (i.v.) push

### 2) Drug Preparation and Schedules

HPPH: PDT was done 24 hours after PS treatment
   Immunotherapy (BCG vaccine)˜2×10⁶ cells in 100 ul/dose subcutaneously (SQ)Chemotherapy: First dose 1 hour post PDT and 2 doses weekly thereafter for weekly schedule for BCG SQ treatments.
   3) Tumor transplantation: The xenografts used for antitumor activity were transplanted subcutaneously. The treatment was initiated 7-10 days post transplantation.
   4) Tumor measurement: Two axes (mm) of tumor (L, longest axis; W, shortest axis) were measured with the aid of a Vernier caliper. Tumor weight (mg) was estimated as a formula: tumor weight=½ (L×W2). Tumor measurements were taken after the scabbing healed post PDT and at least three times a week for post therapy and twice a week therefore.
   5) Tumor regression: Complete tumor regression (CR) was defined as the inability to detect tumor by palpation at the initial site of tumor appearance for more than two months post therapy. Tumor regrowth after CR occurred was observed in less 5% of mice. Partial tumor regression (PR) was defined as 50% reduction in initial tumor size.
   Normally, 5 mice per treatment group were included in these experiment groups and each experiment repeated at least twice.

PDT of Subcutaneous Tumors: Tumor bearing mice are injected via the tail vein with HPPH or derivatives at doses that are non-toxic unless exposed to light. For tail vein injections, mice were gently restrained in approved holders and their tails were briefly (less than 1 minute) dipped in warm sterile water (˜40° C.). Photosensitizers are injected in a volume of less than 0.2 mL into the tail vein using a 27-gauge needle.

The photosensitizers, at 24 hours (or the optimal time determined by optical imaging) after administration of HPPH or derivatives, the animals were partially restrained allowing leg movement, in specially designed holders without anesthesia. The animal restraint procedure has been demonstrated to and approved by Laboratory of animal resources (LAR).

Tumors are exposed to visible light (1 cm diameter) at power densities of less than 100 mW/cm². The light sources are either a dye laser (with a tunable range of 600 to 800 nm), at the optimal excitation wavelength for the individual photosensitizer; or a pulsed laser with a range of 460 to 800 nm. The treatment will vary, depending on the light dose (J/cm²) and fluence rate (mW/cm²) required, but usually is for approximately 30 minutes. The mice are held still in the specially designed holders with no problems during treatment.

After light exposure, the mice are monitored closely for at least 1 hour and then daily until the re-growing tumors reach no more than 2 cm in the greatest dimension or for a maximum of 60 days (post treatment) at which time they are euthanized. All animals will be euthanized within 90 days of tumor implantation.

UMUC-3, T24 (Urinary Bladder). Previous data with PDT in urinary bladder cancer tumors have shown response rates and want to see if the response rates are better in combination with immunotherapy.

The results suggest that HPPH and Photobac in combination with chemotherapy agents: doxorubicin and cisplatin, irinotecan and BCG produce enhanced PDT efficacy in mice bearing lung, head & neck and urinary bladder cancer respectively. The use of highly effective PDT agents with fluorescence-imaging ability in NIR region shows that these PS can be used in image-guided PDT at a low dose. Chemotherapy-alone at a dose that was not effective on administering at the same dose after PDT significantly enhanced the long-term cure. Such an unexpected long-term cure was not observed by the PDT or chemotherapy alone. In addition, in contrast to most of the clinically approved PS, HPPH and Photobac do not produce any skin photo toxicity. Finally, the combination of PDT with a chemotherapy drug of choice could be extremely helpful as a personalized therapy for the cancer patients with limited toxicity and improved quality of life.

### Example 2

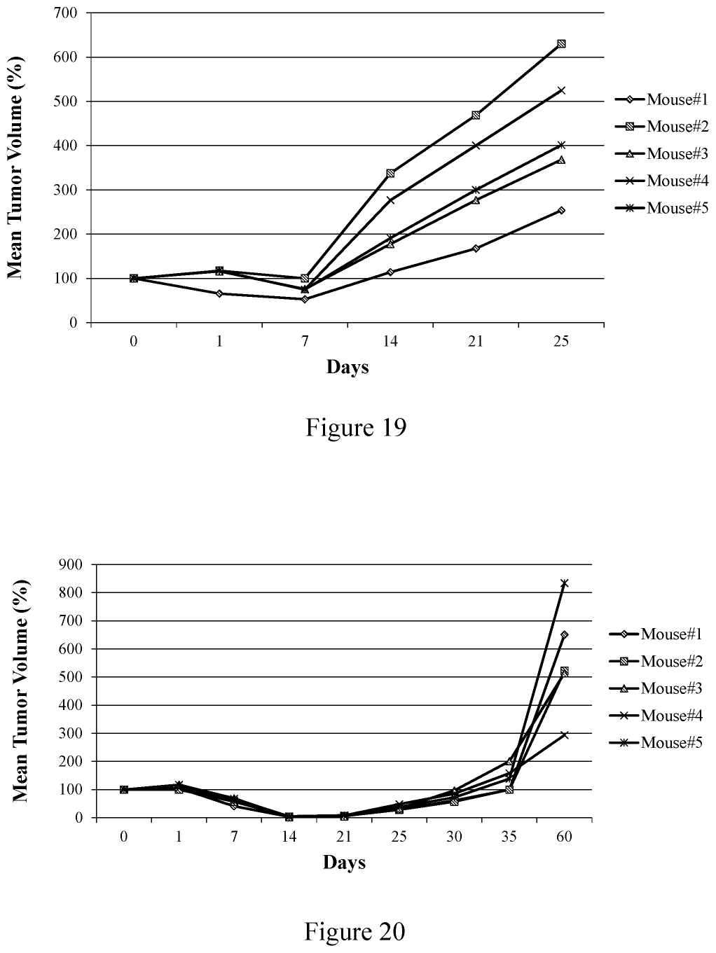

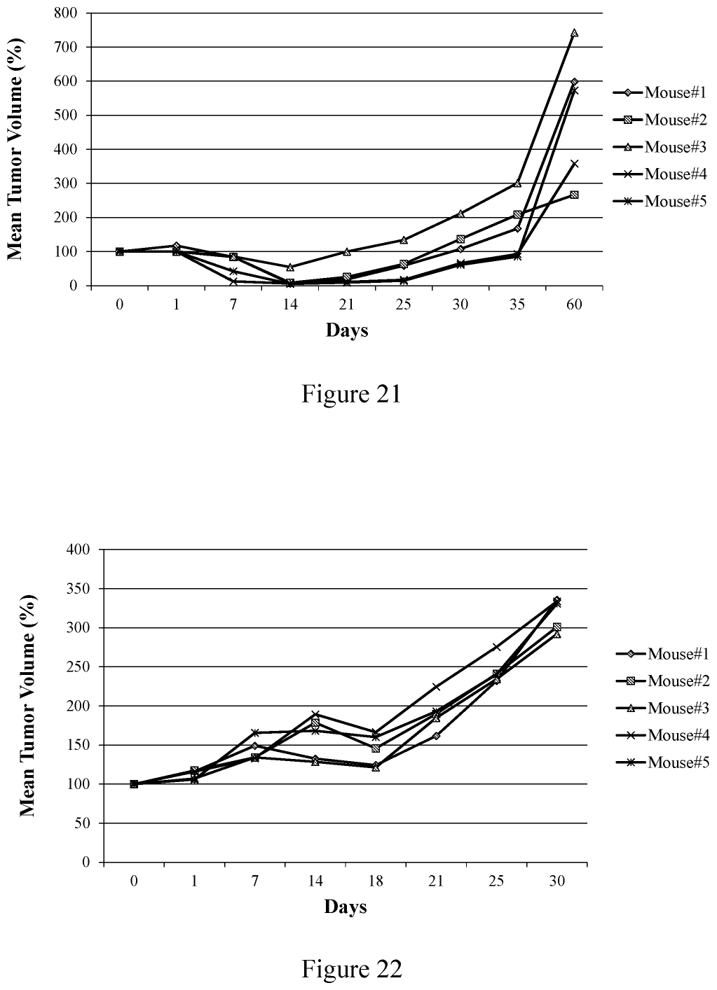

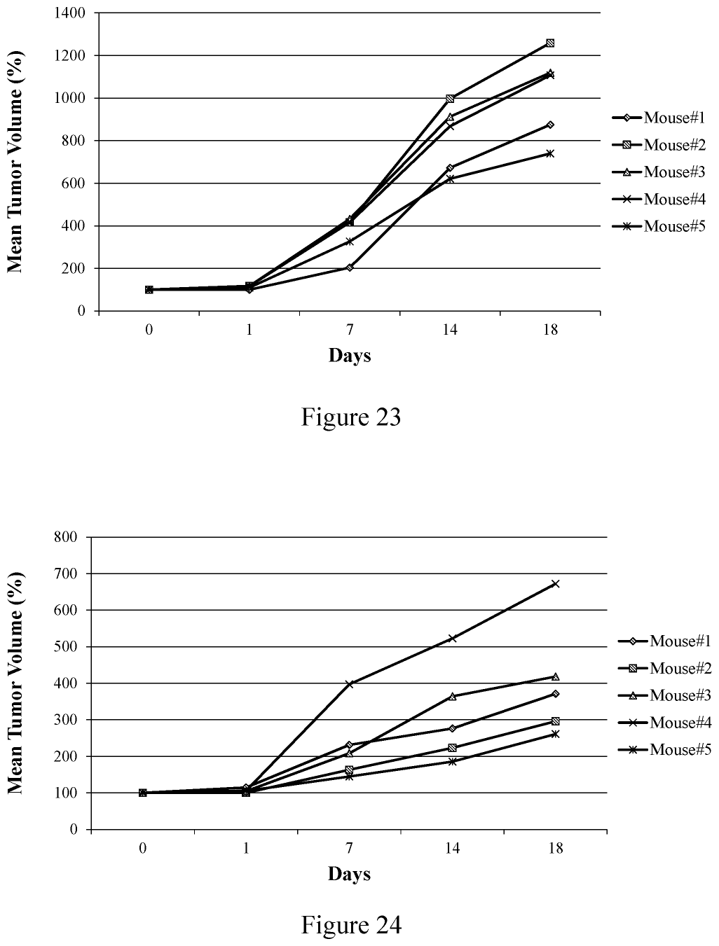

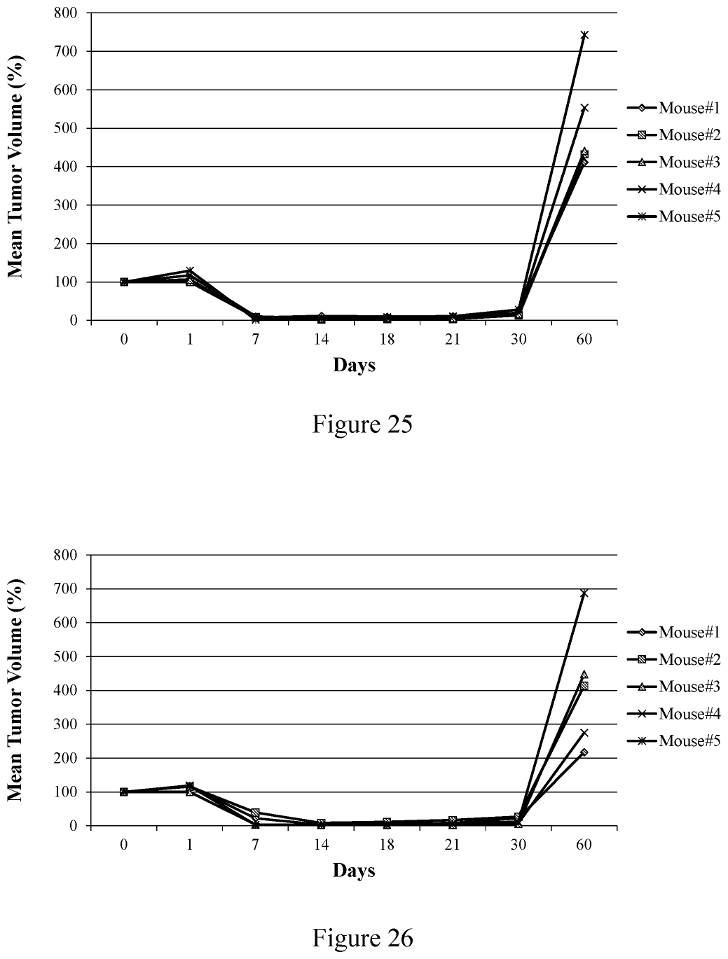

Comparative results on the effect of tumor volume when using chemotherapy agents alone are shown in FIGS. 19-26. The experiments were carried out according to the methods described in Example 1 and in the figures.

Although the present disclosure has been described with respect to one or more particular embodiments and/or examples, it will be understood that other embodiments and/or examples of the present disclosure may be made without departing from the scope of the present disclosure.

1. A method for treating an individual in need of treatment for cancer comprising:
administering to the individual an effective amount of a NIR photosensitizer comprising a tetrapyrrolic core or reduced tetrapyrrolic core;
administering a sub-therapeutic effective amount of a chemotherapy agent; and
irradiating the individual with electromagnetic radiation having a wavelength of 650 nm to 800 nm.

2. The method of claim 1, wherein the NIR photosensitizer is selected from the group consisting of HPPH (3-(1′-hexyloxy)ethyl-3-devinylpyropheopphorbide-a), Photobac (3-(1′-butyloxy)ethyl-3-deacetyl-bacteriopurpurin-18-N-butyl-imide methyl ester), and derivatives thereof.

3. The method of claim 1, wherein the chemotherapy agent is selected from the group consisting of doxorubicin, cisplatin, irinotecan, and combinations thereof.

4. The method of claim 1, wherein the administration comprises i) administration of the NIR photosensitizer, and ii) after completion of the administration of the NIR photosensitizer and irradiation of the individual, administration of the chemotherapy agent.

5. The method of claim 4, wherein the chemotherapy agent is administered 30 minutes to 90 minutes after administration of the NIR photosensitizer.

6. The method of claim 1, wherein the method further comprises visualization of the cancer after administration of the NIR photosensitizer.

7. The method of claim 6, wherein the visualization is carried using fluorescence imaging.

8. The method of claim 6, wherein the method further comprises surgical removal of at least a portion of a cancerous tissue from the individual.

9. The method of claim 1, wherein the cancer is selected from lung cancer, head and/or neck cancer, esophageal cancer, laryngeal cancer, breast cancer, pancreatic cancer, bladder cancer, renal cancer, prostate cancer, testicular cancer, and combinations thereof.

10. The method of claim 1, wherein the effective amount of chemotherapy agent is a sub-therapeutic amount.

11. The method of claim 1, wherein the effective amount of chemotherapy agent is 50% or less of an effective amount of chemotherapy agent necessary in the absence of administration of the NIR photosensitizer.

12. The method of claim 1, wherein the individual is in need of treatment for bladder cancer and BCG is administered after administration of both the NIR photosensitizer and the chemotherapy agent.

13. A pharmaceutical composition comprising one or more NIR photosensitizer and one or more chemotherapy agent.

14. The pharmaceutical composition of claim 13, wherein the pharmaceutical composition further comprises Bacillus Calmette-Guerin (BCG) vaccine.

15. A kit comprising one or more NIR photosensitizer, one or more chemotherapy agent, and instructions for use of the NIR photosensitizer(s) and chemotherapy agent(s) for treatment of an individual.

16. The kit of claim 15, wherein the kit further comprises Bacillus Calmette-Guerin (BCG) vaccine.
